(19) United States
(12) Patent Application Publication
(10) Pub. No.: US 2026/0275051 A1
(43) Pub. Date: Sep. 17, 2026

(54) Utilization of Sugar Beet-Derived Biopolymers for Material Applications

(71) Applicant: University of Wyoming, Laramie, WY (US)

(72) Inventors: Roberta Maia Sabino, Fort Collins, CO (US); Karen E. Wawrousek, Laramie, WY (US); Jenna Crouse, Casper, WY (US); Shad Sellers, Rock Springs, WY (US)

(73) Assignee: University of Wyoming, Laramie, WY (US)

(21) Appl. No.: 19/568,699

(22) Filed: Mar. 16, 2026

Related U.S. Application Data
(60) Provisional application No. 63/772,531, filed on Mar. 15, 2025.

(51) Int. Cl. C08H 99/00; A61L 27/34; A61L 27/54

(52) U.S. Cl. CPC C08H 99/00; A61L 27/34; A61L 27/54; A61L 2420/08

(57) ABSTRACT

Methods and compositions are described for a sugar beet molasses polymer composition produced from sugar beet biomass, including from sugar beet molasses. Polymer compounds and polymer coatings are provided. The polymers can be formulated as coating compositions for use on medical devices. The polymer coatings can be antimicrobial and can have antioxidant properties. The polymer coatings can promote cell adhesion. The polymer can be formulated into coatings, growth media, packaging, cosmetics, personal care products. Formulations including the material can improve biocompatibility, as well as producing antimicrobial, anti-inflammatory, and antioxidant features for health-related methods and products.

### CROSS-REFERENCE TO RELATED APPLICATIONS

This application claims the benefit of U.S. Patent Application 63/772,531 filed on Mar. 15, 2025, the contents of which are incorporated herein by reference in the entirety.

### STATEMENT REGARDING FEDERALLY SPONSORED RESEARCH

N.A.

### BACKGROUND

Polymers can have a wide range of useful properties. Biopolymers can be derived from natural sources to manufacture a wide range of products, including for example, water-resistant coatings, biodegradable plastics, and hydrofracturing additives. Known biopolymers have been developed into products and methods providing useful improvements, and more development is desirable. Accordingly, there is a need for additional methods, compositions, and products utilizing biopolymers.

Studies have shown that an overuse of antibiotics has resulted in the development of numerous antibiotic-resistant strains of bacteria. Some bacteria have even developed tolerance against common disinfectants. As a result, implant infections have become increasingly difficult to treat. Biopolymer-based medical device materials, and implant coatings with antibacterial properties would be advantageous to reduce infections related to biomedical implants.

### SUMMARY

Technology described herein provides biopolymers produced from a botanical source. Provided are methods of making and using SBMP, a biopolymer composition derived from sugar beet.

The present specification generally relates to methods and products using SBMP. SBMP possesses excellent antioxidant and antibacterial activities and is nontoxic to mammalian cells. SBMP can be produced from sugar beet plant material, including from byproducts of a sugar-refining process. SBMP can be used as an antimicrobial additive to aqueous compositions, including cosmetics and healthcare products. SBMP can be used as an antimicrobial coating on medical devices, such as stents, catheters, dental implants, and orthopedic implants. Oligomers and polymers derived from sugar beet processing byproducts can be used as a biomaterial, incorporated into hydrogels, growth media, nanofibers, nanoclays, or tissue growth scaffolds. Provided are compositions of SBMP containing specific chemical moieties with features useful in biomaterial applications.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like reference numerals designate corresponding parts throughout the views.

The patent or application file contains at least one drawing executed in color. Copies of this patent or patent application publication with color drawing(s) will be provided by the Office upon request and payment of the necessary fee.

FIG. 1a is an example MALDI-TOF mass spectra for a SBMP.

FIG. 1b is another example MALDI-TOF mass spectra for the SBMP.

FIG. 2a is a XPS survey scan of the SBMP.

FIG. 2b is a XPS high-resolution spectra for carbon in the SBMP.

FIG. 2c is a XPS high-resolution spectra for oxygen in the SBMP.

FIG. 2d is a XPS high-resolution spectra for nitrogen in the SBMP.

FIG. 3 is a H-NMR spectrum of the SBMP.

FIG. 4 is a C-NMR spectrum of the SBMP.

FIG. 5 is a FTIR spectrum of the SBMP.

FIG. 6 is a SBMP zeta potential with varying pH values.

FIG. 7a is a graph of antioxidant activity of the SBMP at different concentrations (0.25, 0.5, 0.75, 1, 1.25, 1.5, 1.75, and 2 mg/mL), measured as Radical Scavenging Activity (RSA) toward DPPH.

FIG. 7b is a graph of antioxidant activity of the SBMP at different concentrations (0.25, 0.5, 0.75, 1, 1.25, 1.5, 1.75, and 2 mg/mL), reported as ascorbic acid (AC) equivalence.

FIG. 8a is a graph of antibacterial activity of the SBMP at different concentrations (0.63, 0.5, and 1 mg/ml), reported as inhibition of Rhodococcus erthropolis growth.

FIG. 8b is a graph of antibacterial activity of the SBMP at different concentrations (0.63, 0.5, and 1 mg/ml), reported as inhibition of Escherichia coli growth.

FIG. 8c is a graph of antifungal activity of the SBMP at different concentrations (0.63, 0.5, and 1 mg/ml), reported as inhibition of Saccharomyces cerevisiae growth.

FIG. 9a is a graph showing cell viability in different concentrations of the SBMP (0.063, 0.1, 0.25, and 0.5 mg/mL) as determined using an MTT assay after 48 hours of SBMP exposure.

FIG. 9b is a graph showing cell viability in different concentrations of the SBMP (0.063, 0.1, 0.25, and 0.5 mg/mL) as determined using the alamarBlue™ assay after 4 and 8 days of incubation with SBMP.

### DETAILED DESCRIPTION

A biopolymer composition was produced from sugar beet molasses (SBM), and characterized. The presently-disclosed polymer is designated: SBMP. The SBMP was chemically characterized and showed features with beneficial applications in the biomedical sector, as well as other sectors that may use bio-based antimicrobials and/or antioxidants.

Compositions of SBMP are described. Described compositions may be incorporated into cosmetic, medical, healthcare, and therapeutic products. Described compositions may be used in coatings for medical devices. Described compositions may be used as antimicrobial coatings or additives. Coatings or additives using SBMP can be incorporated into medical devices. Methods of forming a biopolymer coating comprising SBMP are provided.

Methods of producing SBMP are provided. In some examples, the feedstock for producing the SBMP comprises sugar beet molasses.

In the United States, approximately 60% of the domestic sugar production comes from sugar beets. In 2023-2024, it is expected that approximately 5 tons of refined sugar will be produced from a sugar beet crop of approximately 33 million short tons. The molasses used as a starting material for this work is a byproduct of sugar production from sugar beets. This sugar beet molasses (SBM) is neither a food product nor a precursor to any food product. It can be used as an animal feed supplement, fertilizer, and a spray for dust control and de-icing roads. The molasses contains any remaining sugars after processing and organic acids including amino acids, betaine, potassium, raffinose, sucrose, sodium, chloride, and nitrates.

SBM includes the presence of tannins and phenolic compounds. These compounds are also detected in sugar beet flesh prior to sugar extraction. Phenolic compounds are secondary metabolites found in plants that can possess bioactive properties such as antioxidant and antimicrobial activity. The SBM may also include lignins which are present in extracted sugar beet pulp. Lignin-derived compounds can produce a reddish hue due to the presence of coniferyl aldehydes. Coniferyl aldehydes are natural phenolic phytochemicals that help defend against radical oxidative species. One application for the biopolymers extracted from SBM is to make the polymers into a coating for medical devices. According to the Centers for Disease Control, indwelling medical devices cause 50% to 70% of the approximately two million healthcare-associated infections. Some of these infections are attributed to bacterial growth and the formation of biofilms. Uncontrolled biofouling can lead to undesired cell ingrowth, infections, and eventually device failure. Compounds in SBM have antimicrobial and antioxidant properties, and the compounds can be incorporated into a coating for limiting microbial growth on medical implants. The compounds can be incorporated as an additive to cosmetic and personal care products, for example, in a solution or emulsion, as an antioxidant or antimicrobial. Another application for the biopolymers and/or oligomers extracted from SBM is in a growth media, such as liquids or gels, for example including hydroponic or tissue growth substrates. Another application for the biopolymers and/or oligomers extracted from SBM is in a tissue engineering scaffold. Materials produced from SBM can make excellent scaffolds due to biocompatibility. Materials produced from SBM can have anti-inflammatory properties. Inflammation hinders tissue regeneration, prolonging recovery times. Biopolymers produced from SBM are non-cytotoxic, and serve as a cheap and safe material for constructing new tissue scaffolds.

SBM was processed to produce the SBMP, and the produced composition was chemically characterized, showing features with beneficial applications in the biomedical sector.

### Experimental Methods

One method to extract and derive the biopolymers from the SBM includes performing a dialysis and lyophilization process. The produced substance is named sugar beet molasses polymer (SBMP) and includes polymers and, in some examples, can further include oligomers. A variety of techniques can be used to chemically characterize the SBMP including Matrix-Assisted Laser Desorption Ionization Time of Flight Mass Spectrometry (MALDI-TOF MS), X-ray photoelectron spectroscopy (XPS), proton and carbon nuclear magnetic resonance (¹H- and ¹³C-NMR), and/or Fourier-transform infrared spectroscopy (FTIR). A zeta analysis can also be conducted to determine the overall charge of the SBMP. The antioxidant and antimicrobial activities, along with the cytotoxicity of the SBMP, can also be analyzed to show the viability to be effectively used for biomedical applications. By using the SBMP as a biomaterial, an agriculture byproduct is transformed into a valuable biomedical asset. The diverse variety of extractable biopolymers, including lignin-derived compounds, tannins, and phenolics, from SBM renders it appropriate for the biomaterial sector.

Utilizing the natural biological properties of plant byproducts to enhance biomedical applications presents the opportunity to combine nature's benefits with sustainable innovation. SBMP, produced from a byproduct of sugar production from sugar beets, has been determined to be suitable for use in biomedical applications, amongst other uses. To characterize the molecules that form the SBMP, MALDI-TOF MS was performed to determine the relative molecular sizes of the SBMP. The chemical composition of the SBMP was also characterized using ¹H-NMR, ¹³C-NMR, XPS, and FTIR. The characterization assays showed that the SBMP comprises phenolic and hydroxide groups. The presence of these groups further indicated the SBMP's high antioxidant activity. The SBMP also displays antimicrobial activity against Rhodococcus erythropolis, Escherichia coli, and Saccharomyces cerevisiae. The SBMP exhibits no toxicity to human adipose derived stem cells (ADSC) at concentrations up to 0.5 mg/mL. SBMP can be used for biomaterial applications, and confer experimentally-demonstrated antimicrobial and antioxidant activity to resulting biomaterial products.

### SBMP Preparation

A dilute solution of SBM in ultrapure water (10 mL SBM in 50 mL water) is dialyzed against ~4.5 L of ultrapure water at 4° C. using a 6-8 kD membrane (Spectra/Por™ part #132665T), with a minimum of 6 water exchanges over a 7-day period. After dialysis, 25 mL aliquots of the dialyzed solution are lyophilized. Approximately 0.12 g of lyophilized sugar beet molasses polymer (SBMP) is recovered from 10 mL of undiluted SBM. Lyophilized SBMP is then stored at room temperature.

### Characterization of SBMP

### Matrix-Assisted Laser Desorption Ionization Time of Flight Mass Spectrometry (MALDI-TOF MS)

The MALDI-TOF MS is performed to determine the relative size of molecules in the SBMP. For data acquisition, SBMP is diluted to 0.5% w/w in ultrapure water and allowed to stir for at least 2 hrs. The diluted SBMP is then combined with 1,6-diphenyl-1,3,5-hexatriene (DPH) matrix to produce solutions that contain 1:1, 1:2, and 1:5 ratios of SBMP:DPH. Then, 1 μL of polyethylene glycol 2,000 (PEG 2,000) is deposited onto a MALDI target plate for calibration purposes. After the calibrant is added, 1 μL of each SBMP:DPH solution is deposited onto the plate and dried under atmospheric conditions for 5 mins for analysis with the Sciex TOF/TOF 5800™ instrument. Each mass spectrum is collected over two ranges: 1) 7,000 to 100,000 m/z and 2) 6,000 to 170,000 m/z. MALDI-TOF MS SBMP dilutions are measured on 5 spots and each spot is read at least three times. The mass spectra data is then analyzed with TOF/TOF™ Series Explorer Software, mMass open-source software, and Origin Software.

### X-Ray Photoelectron Spectroscopy (XPS)

The chemical composition of the SBMP is characterized by X-ray photoelectron spectroscopy (XPS, Shimadzu). Prior to analysis, a thin layer of SBMP powder is adhered to carbon tape on an XPS sample bar. The sample bar is then incubated in a vacuum oven for 24 hrs to remove any volatile components from the SBMP. Survey scan spectra is obtained from 0 to 1200 eV at a pass energy of 80 eV. High-resolution spectra is then collected for carbon (Cls), oxygen (O1s), nitrogen (Ns), silicon (Si2p), and sulfur (S2p). All the high-resolution scans are collected with a pass energy of 40 eV. The XPS scans are calibrated with the binding energy of carbon (284.5 eV). XPS analysis is tested on at least 3 spots on three different powdered samples. MultiPak and Origin software are used for peak-fit analysis.

### Nuclear Magnetic Resonance (NMR)

1H and 13C-NMR spectra are collected to determine the different chemical bonds present in the SBMP. For the acquisition of both spectra, the SBMP is diluted to 10% w/w in D20 and allowed to stir for ~2 hrs. The samples are then added to Norell® Standard Series™ 5 mm NMR tubes prior to analysis. The solution-state NMR spectroscopy data is acquired with a Bruker Avance III 600 NMR spectrometer operating at Larmor Frequencies of 600.2 MHz (H) and 150.9 MHz (C) and a Bruker 5 mm PABBO BB-1H/D Z-GRD probe at 25° C. and Topspin 3.2 (Bruker). The one-dimensional 1H-NMR spectrum is recorded with 30° flip angle, 65536 data points, 128 scans, 20 ppm sweep width, and 1 sec relaxation delay. The one-dimensional 13C-NMR spectrum is recorded with 30° flip angle, 65536 data points, 27648 scans, 300 ppm sweep width, 2 sec recycle delay, and proton Waltz-16 decoupling. After acquiring the scans, the data is analyzed using Origin software.

### Fourier Transform Infrared (FTIR) Spectroscopy

Further analysis of the different functional groups present in the SBMP are analyzed using Fourier transform infrared spectroscopy (FTIR). The FTIR spectra are obtained with the Alpha II FTIR spectrometer (Bruker, Serial #113127) in the spectral range of 400-4000 cm⁻¹, with 10 scans, and a resolution of 4 cm⁻¹. For analysis, a small sample (~1 mg) of lyophilized SBMP is used. The data is analyzed using OPUS software with a peak threshold of 95%, and the data is graphed in Origin.

### Zeta Potential

Zeta potential, a measurement of the electrical potential at the slipping plane of molecules in a solution, is measured using phase analysis light scattering. A stock solution is prepared by diluting the SBMP in ultrapure water to make a 0.1% w/w solution. The stock solution is then stirred for ~2 hrs before further use. After stirring, the stock solution is diluted with either 0.01 M HCl or 0.01 M NaOH to create seven 0.01% w/w solutions with varying pHs (3, 4, 5, 6, 7, 8, 9). The solutions are placed in 1 cm cuvettes and analyzed in a NanoBrook 173 Plus (Brookhaven Instruments, Serial #270005). Data is collected over 1 second, and data analysis is completed in the Particle solution v.3.3 software (Brookhaven Instruments). The zeta potential acquisition is repeated three times for each pH value. For all the solutions, ultrapure water is used for the background measurement.

### Antioxidant Activity

The antioxidant activity of SBMP is analyzed using the free radical 2,2-diphenyl-1-picrylhydrzyl (DPPH) assay, except methanol is replaced with ethanol. The DPPH stock solution is prepared by dissolving 10 mg of solid DPPH (Lot #U101018) in 25 mL of ethanol. The SBMP is dissolved in ultrapure water and is stirred for ~2 hrs before eight serial dilutions are made (0.25, 0.5, 0.75, 1, 1.25, 1.5 1.75, and 2 mg/mL). In the dark, 100 μL of the stock DPPH stock solution is added to 100 μL of a SBMP solution in a 96 well plate. This process is repeated for each SBMP serial dilution. In the same well plate, 100 μL of ethanol is added to 100 μL of a SBMP solution. This process is also repeated for each SBMP serial dilution. These wells are used as blanks to account for the background color of the SBMP. 100 μL of the DPPH stock solution plus 100 μL of ultrapure water is used as the control. The plate is incubated in the dark on a shaker plate (235 rpm) for 30 minutes. After 30 minutes, the absorbance of the plate was read at 517 nm using a Biotek H1 Synergy microplate reader. The DPPH assay is repeated three times with 5 replicate measurements for each test condition. The background absorbance is repeated three times with 3 replicate measurements. Ascorbic acid (AC) is used as the reference standard. Five AC solutions (1, 50, 100, 150, and 200 mg/mL) are prepared in ultrapure water and are used to build a standard curve. The antioxidant activity of the SBMP is analyzed using AC equivalents (mg/mL) and radical scavenging activity (RSA). The RSA is calculated using RSA (%)=[C₅₁₇−A₅₁₇/C₅₁₇]×100. Where, C₅₁₇ is the average absorbance of the control at 517 nm and A₅₁₇ is the absorbance of the diluted SBMP DPPH solution minus the corresponding average background reading of the SBMP dilution at 517 nm.

### Microbial Growth Inhibition

For the growth inhibition (GI) assays, one gram-positive bacteria (Rhodococcus erythropolis), one gram-negative bacteria (Escherichia coli), and one fungus (Saccharomyces cerevisiae) were used. Prior to analysis, the bacterial liquid growths were prepared in M9 medium containing 0.4% glucose, and the fungal liquid growth were prepared in synthetic complete dextrose (SCD) medium (1.7 g yeast nitrogen base, 5 g ammonium sulfate, 20 g dextrose, amino acid mix). The R. erythropolis and S. cerevisiae cultures are incubated at 30° C., shaking at 150 rpm, for 24 hrs prior to use in the GI assay, and the E. coli cultures were incubated at 37° C., shaking at 150 rpm, for 24 hrs prior to use in the assay. The microbial growth inhibition assays are based off the guidelines of the Clinical and Laboratory Standard Institute using the media microdilution method with M9 and SCD media. The microbial suspensions of R. erythropolis and E. coli are created by diluting the microbial solutions to an optical density of 0.01. The optical density of the microbial growths were measured at 595 nm using a NanoDrop 2000c Spectrometer (Serial #Q685). The microbial suspension of S. cerevisiae is created by diluting the microbial solution to ~1×10⁶ cells/mL. In 96-well plates, 100 μL of diluted microbial solutions are added to 100 μL of three different SBMP solutions in ultrapure water (0.063, 0.5, and 1 mg/mL). The 96-well plates also include wells that contain 100 μL of diluted microbial solutions and 100 μL of ultrapure water (positive control) and wells with 100 μL of SCD or M9 media and 100 μL of ultrapure water (negative control). Due to the color of the SBMP, wells to account for the background are prepared with 100 μL of the three different SBMP concentrations and 100 μL of SCD or M9 media. Once the plates are prepared, the initial optical density of each well was read at 595 nm using a Biotek Synergy H1 Microplate reader. After the initial reading, the plates are wrapped in parafilm and placed in separate incubators with water dishes at the bottom. The parafilm and water dishes are added to reduce evaporation of the liquid inside the plates. The R. erythropolis and S. cerevisiae plates are incubated at 30° C. for 24 hrs, and the E. coli plates are incubated at 37° C. for 24 hrs. After 24 hrs, the absorbance of each well was measured at 595 nm. The percent growth inhibition for each SBMP concentration is calculated using Growth Inhibition (%)=(1-A₅₉₅/(C_{pos,595}-C_{neg,595}))×100. Where, A₅₉₅ is the absorbance of the culture mixed with SBMP minus the average SBMP background reading for that SBMP concentration at 595 nm, C_{pos,595} is the average absorbance of the positive control, and C_{neg,595} is the average absorbance of the negative control. All the growth inhibition studies are repeated at least three times with 5 replicate measurements for each test condition and background reading.

### Cell Viability

Human adipose derived stem cells (ADSC) are used for both the 3-(4,5-dimethylthiazol-2-yl)-2,5-diphenyltetrazolium bromide (MTT) and alamarBlue™ assays. ADSC are cultured in 75 cm² surface area tissue-culture polystyrene flasks using MEM growth media (MEM Alpha Modification, ThermoFisher) containing 10% fetal bovine serum and 1% penicillin/streptomycin. The cells are then incubated at 37° C. in a 5% CO₂ atmosphere. For the following assays, all the ADSC used are below passage seven.

The MTT assay is conducted to determine the cytotoxicity of the SBMP, and the alamarBlue™ assay is conducted to measure cellular proliferation in the presence of SBMP. Prior to running an assay, an SBMP stock solution is prepared by diluting SBMP in cell growth media to a concentration of 1.2 mg/mL. The stock solution is then allowed to stir for ~2 hrs. The SBMP stock solution is then sterilized by filtration (0.2 μm, ThermoFisher). The SBMP stock solution is then diluted to the following concentrations: 0.063, 0.1, 0.25, and 0.5 mg/mL SBMP. For both the MTT and alamarBlue™ assays, 500 μL of each SBMP dilution are combined with 100 μL of a 4.0×10⁴ cells/mL ADSC solution in a 24 well plate. The plates also contain wells with 500 μL of the cell growth media and 100 μL of a 4.0×10⁴ cells/mL solution (positive control). The final cell concentration in each well is 6.7×10³ cells/mL. The negative control wells contain only 600 μL of the cell growth medium. The background absorbance of the SBMP is also accounted for by preparing plates that contain the corresponding SBMP concentrations dissolved in ultrapure water. The CyQYANT™ MTT Cell Viability Assay Kit is purchased from ThermoFisher and is performed according to the manufacturer's protocol with minor modifications. For the MTT assay, the plates are incubated for 48 hrs before the experiment is conducted. After 48 hrs, 60 μL of 12 mM MTT stock solution is added to each well in the dark. The plates are protected from light from this point on and placed back in the incubator for four hours. Then, 600 μL of the SDS-HCl solution is added to each well and the plates are incubated for four more hours. The absorbance of each plate is then measured at 570 nm and 650 nm using a Biotek H1 Synergy microplate reader. The cytotoxicity of the different SBMP concentrations is calculated using Cytotoxicity (%)=([(A₅₇₀−A₆₅₀)−(C_{neg,570}−C_{neg,650})]/[(C_{pos,570}−C_{pos,650})−(C_{neg,570}−C_{neg,650})])×100. Where, A₅₇₀ is the absorbance of the diluted SBMP with ADSC at 570 nm minus the corresponding average SBMP background reading at 570 nm, A₆₅₀ is the absorbance of the diluted SBMP with ADSC at 650 nm minus the corresponding average SBMP background reading at 650 nm, C_{neg,570} is the average absorbance of the negative control at 570 nm, C_{neg,650} is the average absorbance of the negative control at 650 nm, C_{pos,570} is the average absorbance of the positive control at 570 nm, and C_{pos,650} is the average absorbance of the positive control at 650 nm.

For the alamarBlue™ assay, 24-well plates containing ADSC and SBMP are prepared as described above. Plates are incubated for 4 days at 37° C. in a 5% CO₂ atmosphere, and then 60 μL of alamarBlue™ HS Cell Viability Reagent (Lot #2747532, ThermoFisher) is added to each well in the dark. The plates incubated for an additional 4 hrs at 37° C. in a 5% CO₂ atmosphere. The absorbance of each well is then measured at 570 nm and 600 nm using a Biotek H1 Synergy microplate reader (Serial #14020714). The chemical reduction of alamarBlue™ was calculated using Reduction of alamarBlue™(%)=([(E_{oxi,600}×A₅₇₀)−(E_{oxi,570}×A₆₀₀)]/[(E_{red,570}×C_{neg,600})−(E_{red,600}×C_{neg,570})])×100. Where E_{oxi,570} is 80586, E_{red,570} is 155677, E_{oxi,600} is 117216, E_{red,600} is 14652, A₅₇₀ is the absorbance of the diluted SBMP with ADSC at 570 nm minus the corresponding average absorbance of the SBMP background at 570 nm, A₆₀₀ is the absorbance of the diluted SBMP with ADSC at 600 nm minus the corresponding average absorbance of the SBMP background at 600 nm, C_{neg,570} is the average absorbance of the negative control at 570 nm, and C_{neg,600} is the average absorbance of the negative control at 600 nm. After the plates are read, the media is aspirated. The media is then replaced in each well and plates are placed back in the incubator for four more days. The previously explained alamarBlue™ procedure is repeated and the reduction of alamarBlue™ is recalculated to determine cellular proliferation over time. All the cell studies are repeated two times with four replicate measurements for each test condition. The background readings are repeated three times with four replicate measurements.

### Statistical Analysis

GraphPad Prism software is used to perform analysis of variance (ANOVA) and Tukey tests. The statistical differences are compared using (*) for p≤0.05, (**) for p≤0.01, (***) for p≤0.001, and (****) for p≤0.0001.

### Experimental Results

### Characterization of SBMP

### Matrix-Assisted Laser Desorption Ionization Time of Flight Mass Spectrometry (MALDI-TOF MS)

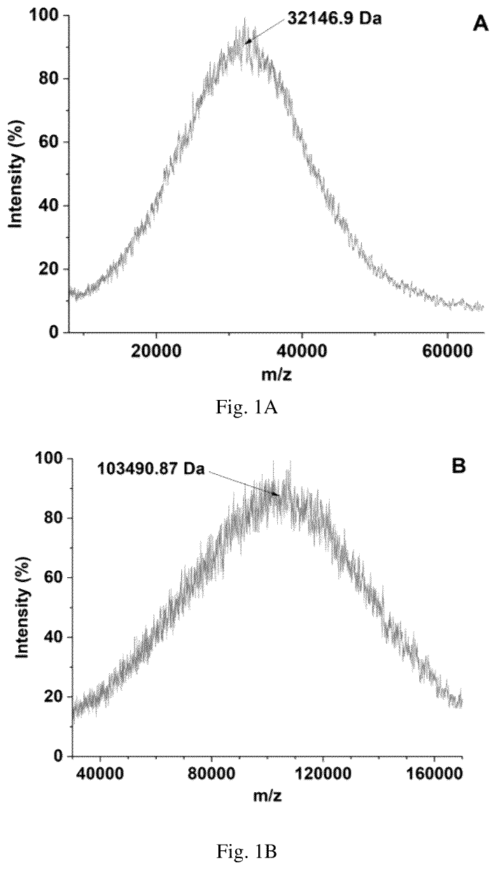

One of the key advantages of using MALDI-TOF MS to analyze polymers is it can measure the average molecular weight. Biopolymers are generally large molecules that can have sizeable variations in their molecular weights. Due to the expected large mass range and possible size variation of constituents in SBM, MALDI-TOF spectra of SBMP were collected over two ranges: 7,000-100,000 m/z and 6,000-170,000 m/z. Prior to analyzing the data, the data was denoised with a 15-point Gaussian filter to reduce the effects of the background noise. FIG. 1A shows the MALDI-TOF spectrum from the first range with a peak located between 10,000 to 55,000 m/z, with the apex of the peak located at 32,146.9 m/z. The peak had a Mw (weighted average molecular weight) of 33,759.1 g/mol, a Mn (number average molecular weight) of 30,622.5 g/mol, and a polydispersity index (PDI) of 1.1024. For a polymer, the PDI can be less than 1.2 for MALDI results to be considered reasonable. With a PDI of 1.1024, the SBMP has a low enough PDI for the molecular weight estimations to be accurate. A second peak was detected between 35,000 and 165,000 m/z (FIG. 1B). The apex of the peak was located at 103,490.87 m/z. The peak had a Mw of 107,517.2 g/mol and a Mn of 97,653.8 g/mol. The PDI for the second peak was 1.1046, which is also below the 1.2 limit. This peak's broadness shows a wide distribution of different sized macromolecules. Beside natural variance, the variation in the SBMP sizes may be attributed to environmental degradation (temperature, humidity, oxygen, light, etc.) or physical reactions with other compounds. X-ray Photoelectron Spectroscopy (XPS)

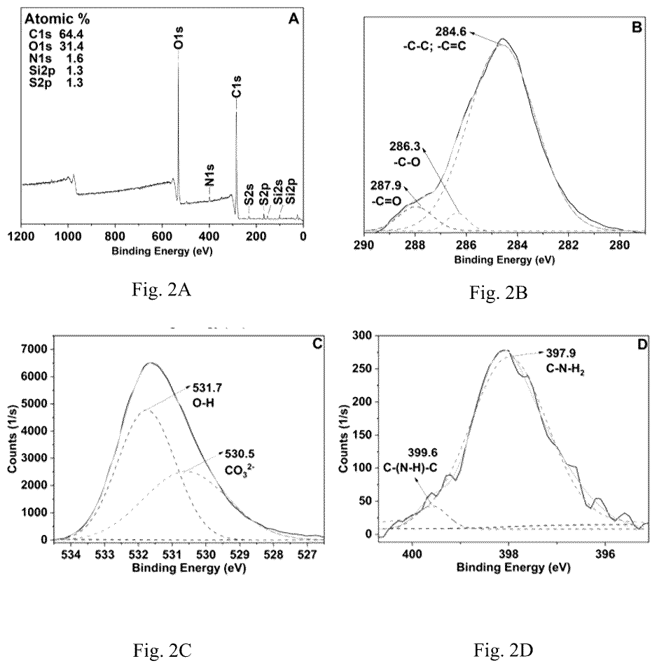

XPS scans were collected to analyze the elements present in SBMP as well as different covalent bonds present in the SBMP. To accomplish this, both a survey scan (FIG. 2A) and three high resolutions scans (FIGS. 2B-D) were performed. The survey scan contains seven peaks, indicating the SBMP is primarily composed of O (O1s at 531.3 eV), N (N1s at 399.0 eV), and C (Cls at 284.7 eV). Of these, carbon comprised the largest percent area of 64.4% in the scan. Small amounts of S (S2s at 231.9 eV and S2p at 168.0 eV) and Si (Si2s at 152.1 eV and Si2p at 101.4 eV) were detected. Silicon and sulfur only comprised 1.3% of the total percent area of the peaks, indicating that a very small amount of these elements or that these elements are contaminants. High-resolution scans of both Si and S confirmed that these elements were present at levels consistent with contaminants, and were excluded from future analysis.

The carbon high-resolution scan showed the presence of carbon-carbon single bonds and carbon-carbon double bonds at 284.6 eV, carbon-oxygen single bonds at 286.3 eV, and carbon-oxygen double bonds at 287.9 eV (FIG. 2B). The oxygen high-resolution scan further supports these conclusions by showing the presence of carboxyl groups (530.5 eV). The high-resolution scan also showed the presence of hydroxide (531.7 eV) and carboxyl groups, represented by the CO3 in the scan (FIG. 2C). Lastly, the high-resolution nitrogen scan showed the presence of primary and secondary amines (397.9 eV) (FIG. 2D). The detected chemical bonds from the XPS high-resolution scans suggest the presence of carboxylic acids, amine groups, hydrocarbon chains, and hydroxide groups.

### Nuclear Magnetic Resonance (NMR)

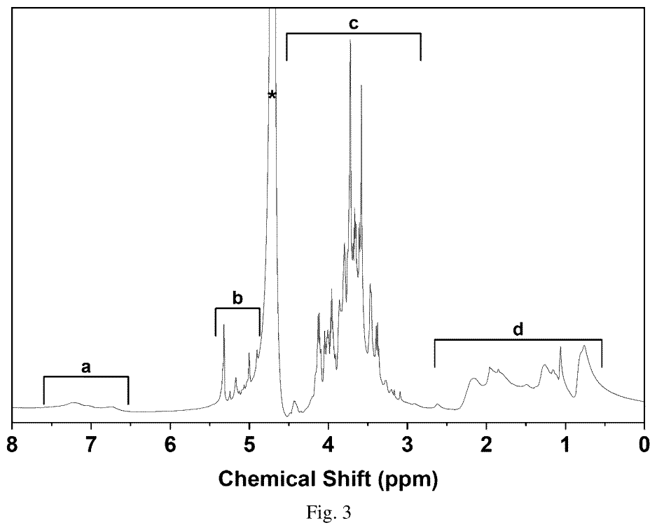

The ¹H-NMR spectrum of the SBMP had four distinct regions containing peaks (FIG. 3). Region a (6.7 to 7.6 ppm) contains two broad peaks that are consistent with the chemical shift of protons in aromatic rings. The peaks in region b (4.9 to 5.6 ppm) represent protons connected to double-bonded carbons and aldehyde protons. The peaks in region c (2.7 to 4.4 ppm) correspond to a multitude of protons in different chemical bond environments, including amine protons, heterocyclic protons, protons bonded to carbon with an oxygen single bond, and protons adjacent to double bonded carbons. The peaks in region d (0.1 to 2.7 ppm) are within the chemical shift range of saturated hydrocarbon protons. The peak denoted with an asterisk (4.8 ppm) was not included in the percent area calculations because it was produced by the solvent D20. The H-NMR spectra confirmed the presence of various bonds identified in the high-resolution XPS scan. The hydroxide group present in the XPS oxygen high-resolution scan was not confirmed with the H-NMR spectra since D20 was used as the solvent. The detected proton environments were not those expected for tannins, polyphenolics, and lignin breakdown products. However, these compounds may have undergone significant chemical reactions during processing for sugar extraction. Alternatively, there can be other compounds present in sugar beets that form biopolymers.

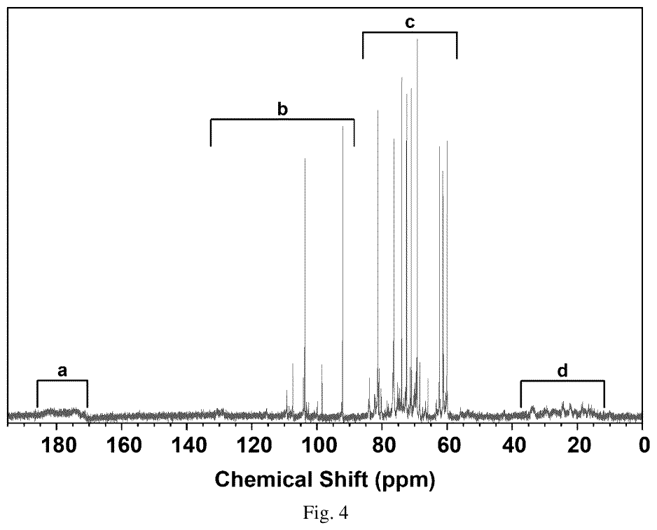

The ¹³C-NMR spectrum of the SBMP was also separated into four distinct peak-containing regions (FIG. 4). Region a (174.0 to 182.1 ppm) contains peaks that represent carboxyl and phenol groups. The peaks in region b (92.1 to 130.7 ppm) represent double-bonded carbons and carbons in aromatic rings, while those in region c (60.1 to 83.9 ppm) correspond with carbon-oxygen single bonds and carbon-nitrogen single bonds. Peaks in region d (16.5 to 33.7 ppm) represent carbons in saturated hydrocarbon chains. The high-resolution XPS scans confirmed all the bonds present in the C-NMR spectra. Higher chemical shifts are associated with electron-withdrawing environments, while lower chemical shifts are associated with electron-donating environments. Based on this, the presence of specific chemical groups such as carboxyl (withdrawing) and amines (donating) are indicated.

Both the H-NMR and C-NMR spectra confirm the presence of aromatic rings, amines, carbon-carbon double bonds, carbon-oxygen single bonds, and saturated/unsaturated hydrocarbon chains. The H-NMR confirms the presence of polysaccharides due to the abundance of narrow peaks within the 3.0 to 5.5 ppm range. The conglomeration of polysaccharides in the solution would contribute to the SBMP's high molecular weight. The functional groups identified from both spectra also shows the presence of lignins. Like lignins, the SBMP contains aromatic rings, carbon-oxygen single bonds, carbon-carbon double bonds, and methoxylated groups. Lignins also have phenylpropanoids, a group of molecules with aromatic carbon rings and oxygen groups, which are present in the SBMP.

### Fourier-Transform Infrared (FTIR) Spectroscopy

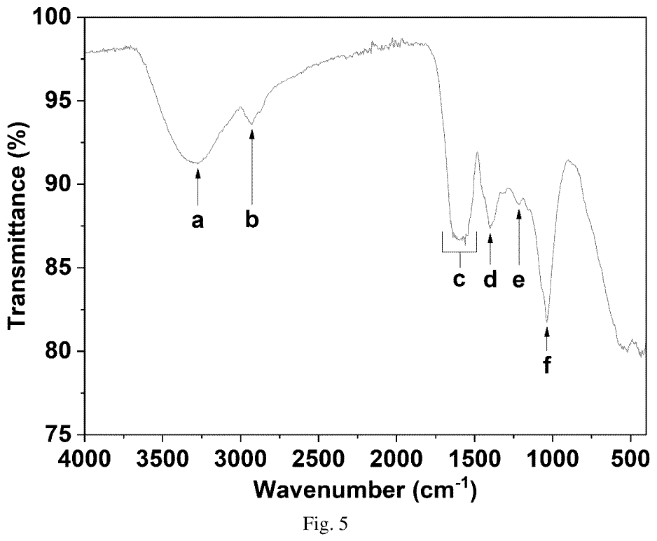

FIG. 5 shows the FTIR spectrum for the SBMP. The spectrum contains six main peaks excluding the fingerprint region. The first region (3000-3750 cm⁻¹) contains peak a which is located at 3275.6 cm⁻¹. This broad peak represents hydroxyl groups including the ones present in phenolic compounds. The second region (2500-3000 cm⁻¹) contains peak b which is located at 2929.0 cm⁻¹. This peak represents carbon-hydrogen and oxygen-hydrogen single bond stretching present in alkanes and carboxylic acids. The third region (1500-1750 cm⁻¹) contains three peaks (1560.7 cm⁻¹, 1596.6 cm⁻¹, and 1637.7 cm⁻¹) all labelled c on the spectrum. The peaks at 1560.7 cm⁻¹ and 1596.6 cm⁻¹ represent aromatic carbon-carbon double bond stretching and amine nitrogen-hydrogen single bond bending. These peaks could also represent asymmetric and symmetric COO-stretching. The peak at 1637.7 cm⁻¹ represents carbon-oxygen double bond stretching. The fourth region (1100-1500 cm⁻¹) contains peak d at 1399.9 cm⁻¹ and peak e at 1214.1 cm⁻¹. These peaks represent carbon-hydrogen single bond stretching. The last region (750-1100 cm⁻¹) contains peak f at 1036.7 cm⁻¹ which represents a carbon-oxygen single bond in an ester. The detected functional groups and bonds were confirmed with the other chemical composition scans. The XPS carbon high resolution scan confirmed the presence of alkanes, alkenes, carbon-oxygen double bonds, and carbon-oxygen single bonds. The XPS oxygen high resolution scan confirmed the presence of hydroxyl groups, carbon-oxygen double bonds, and carbon-oxygen single bonds. The XPS nitrogen high resolution scan confirmed the presence of amines. The H-NMR spectra confirmed the presence of alkanes, alkenes, amines, aromatic rings, carbon-oxygen double bonds, and carbon-oxygen single bonds. The C-NMR spectra confirmed the presence of phenolic groups, alkanes, alkenes, aromatic rings, amines, carbon-oxygen double bonds, and carbon-oxygen single bonds. The FTIR spectrum for the SBMP contains similarities to FTIR spectra of polysaccharides, lignins, and tannins. The three compounds share the broad hydroxyl peak with the SBMP. For lignins and tannins, this peak also represents the hydroxyl groups present in their phenolic compounds. These groups also share peak b with the SBMP which represents the carbon-hydrogen single bond stretching in each compound. Both tannins and lignins also share the multiple peak region (1400-1600 cm⁻¹) with the SBMP which represents the carbons in aromatic rings. The three compounds also share peaks within the 1000-1300 cm-1 range which represent the carbon-oxygen single bond stretching. The FTIR spectrum of the SBMP provides additional evidence that its chemical composition closely resembles lignins, tannins, and polysaccharides.

### Zeta Potential

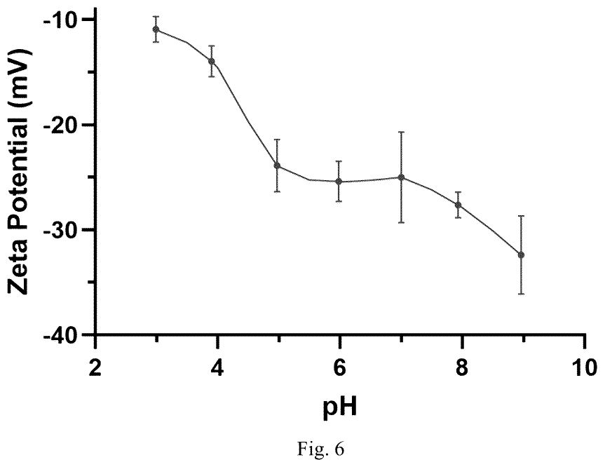

Zeta potential was measured as a surface charge of the SBMP. Measuring zeta potential as a function of pH revealed zeta potentials ranging from −10.9 mV at pH 3 to −32.4 mV at pH 9 (FIG. 6). Between a pH of 5.5 and 7, the SBMP reached a stable zeta potential of ~25.3 mV. The overall trend in FIG. 6 is consistent with other weakly acidic polymers. At lower pH, the ratio of [HA] to [A⁻] is high because the acidic environment favors protonation. As the pH increases, the ratio of [A⁻] to [HA] increases due to the deprotonation of the acid. The data in FIG. 6 was also used to calculate Zetamax which was used to find the pKa values of the SBMP. Two distinct pKa values were found: ~3.6 and ~10.1. The first pKa value corresponds to the pKa of carboxyls (pKa 3-4) and the second pKa value corresponds to the pKa of phenols (~10). These functional groups were confirmed by FTIR, XPS, and NMR.

For biomedical applications, the charge of the SBMP is relevant for determining how it can be used. For example, in layer-by-layer (LbL) deposition of polyelectrolyte multilayers (PEM) coatings, alternating thin deposits of polycation and polyanion layers are added onto a charge surface. The electrostatic interactions of the layers help form a solid coating. PEM coatings are designed to promote or prevent cell adhesion and inhibit bacterial growth by adding a bioactive compound as the terminal layer. Cell proliferation or antibacterial activity of the SBMP indicates suitability as a LbL PEM coating. Since the SBMP has an overall negative charge, it can be used as the polyanion layer for the PEM coating. The overall negative surface charge is likely due to the presence of carboxyl and hydroxide groups.

### Antioxidant Activity

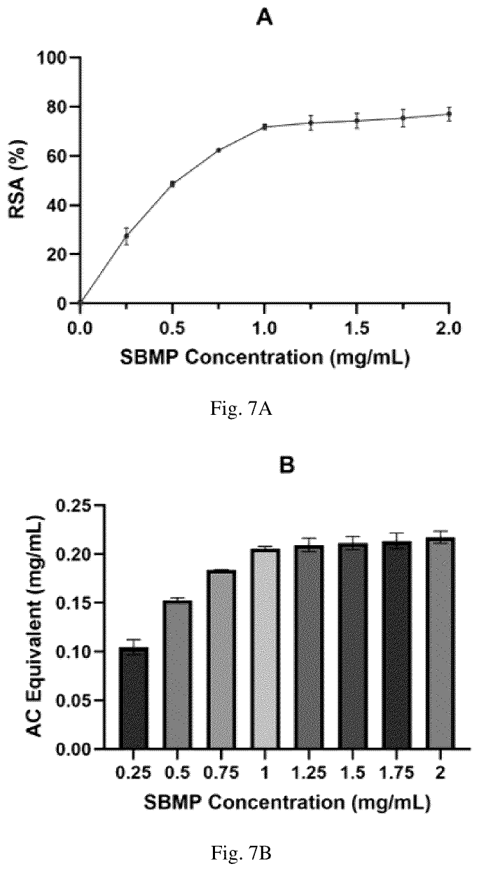

Antioxidants are known for their ability to protect cells against free radicals, which play a role in the development of different conditions such as heart disease, inflammation, and cardiovascular diseases. Natural antioxidants are generally plant derived, and an extract of sugar beet peels includes high antioxidant activity. Antioxidant activity of the SBMP was assessed by measuring oxidation of the chemical DPPH in the presence and absence of SBMP, and the measured radical scavenging activity (RSA) was compared to that of ascorbic acid. Radical scavenging activity increased with SBMP concentration until ~80% RSA was achieved in solution (FIG. 7A). The ascorbic acid equivalence for each SBMP concentration was calculated using the standard curve (R2-0.968). The SBMP at 1 mg/mL or higher concentration has an RSA equivalent of 0.22 mg/mL ascorbic acid (FIG. 7B). The RSA of SBMP is relatively high compared to other plant extracts. Other reported plant extracts RSAs are much lower such as diluted Ficus religiosa leaf extract which has an RSA of ~43%. Dalbergia sissoo leaf extract, which contains tannins and coumarins, has a slightly higher RSA (86.3%) than the SBMP. Dalbergia sissoo's high RSA is attributed to presence of tannins, and tannins in the SBMP contribute to its high RSA. Lignins, which also are present in the system, also have high antioxidant activity due to their phenolic groups. The methoxyl groups and conjugated double bonds in lignins help stabilize phenoxyl radicals which improves the overall antioxidant properties of lignins. Extracted organosolv lignins from eucalyptus have almost an equivalent RSA to the SBMP of ~79%. Like these high RSA extracts, SBMP is contains lignins and tannins, which contain phenolic rings. Additionally, the high RSA also are attributed to the presence of carboxyl and amine groups, as both functional groups are good radical scavengers that would enhance the antioxidant activity of the SBMP.

### Microbial Growth Inhibition

The gram-negative bacterium E. coli is commonly found in the human gastrointestinal tract; however, outside the gastrointestinal tract it can result in extraintestinal illnesses. E. coli is also known for its ability to form biofilms on medical devices which can result in implant failure. Medical implant-associated infections have also occurred with fungi and gram-positive bacteria. Pathogens in biofilms have higher resistances to a host's immune response and higher tolerances towards antibiotics. Due to these potential risks, it is advantageous for an implanted medical device or scaffold to contain a coating with strong antimicrobial properties.

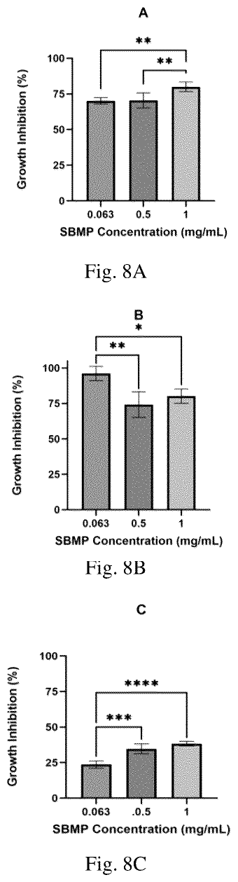

The antimicrobial properties of SBMP toward a gram-negative bacterium, a gram-positive bacterium, and a fungus were investigated, with microbial growth inhibition measured in the presence of 0.063, 0.5, and 1 mg/mL SBMP. SBMP inhibited growth of both the gram-positive bacterium, Rhodococcus erythropolis, and the gram-negative bacterium, E. coli, in liquid culture (FIG. 8A, B). Both R. erythropolis and E. coli had roughly the same amount of GI (~80%) at a SBMP concentration of 1 mg/mL. While SBMP did inhibit some growth of the yeast Saccharomyces cerevisiae (FIG. 8C), growth was inhibited by 38% in the presence of 1 mg/ml SBMP, indicating the SBMP has stronger antibacterial properties than antifungal properties. The antimicrobial activity of the SBMP is attributed to the chemical moieties present in tannins and lignin, with presence of phenolic, polyphenol, terpenoids, alkaloids, and hydroxide groups which are found in tannins. Bioactive plants extracts contain a mixture of these groups, and they can inhibit several bacterial mechanisms resulting in protein inactivation, decreased membrane integrity, efflux pump inhibition, and disruption of biofilm formation. Lignins also contain antifungal properties. Extracted lignins from organosolv spruce displayed GI (50-75%) against Aspergillus niger compared to extracted lignins from kraft spruce which displayed GI (75-100%). The difference between GI percentages was attributed to the higher percentage of carbohydrates in the oganosolv spruce lignins (9.7%) compared to the kraft spruce lignins (2.7%). The presence of polysaccharides in the SBMP also decreases its antifungal activity. The presence of the antibacterial and antifungal activity from the SBMP hinders microbial growth on medical implants.

### Cell Viability

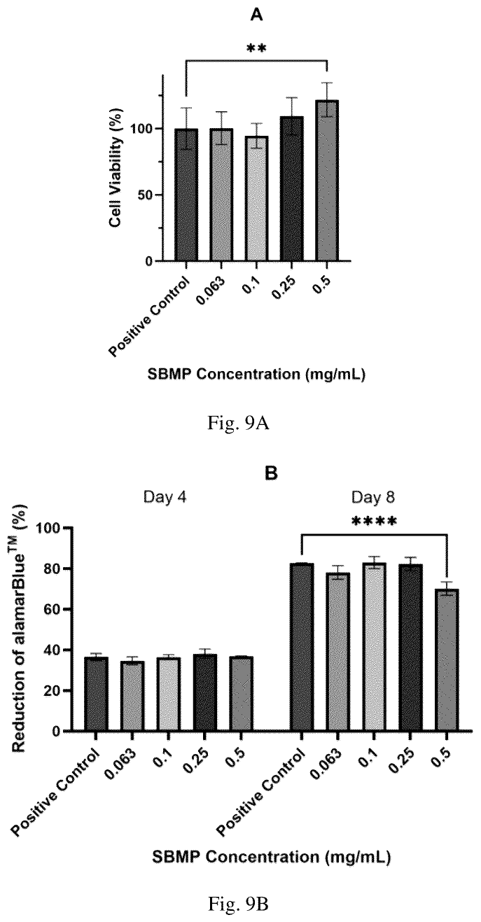

For use in biomedical coatings, a material cannot cause cell lysis or cell death of human cells. Two different assays were used to assess the cytotoxicity and cell viability of SBMP, the MTT assay and the alamarBlue™ assay. In the MTT assay, a water-soluble yellow tetrazolium salt is easily taken up by viable cells. Metabolically active cells reduce the tetrazolium salt into formazan (a purple color) using mitochondrial succinate dehydrogenase. SBMP impacts on cellular viability is assessed by measuring produced formazan in the presence of SBMP and comparing it to the formazan produced by untreated cells. If cell viability decreases to less than 70% in the presence of a compound, it is deemed cytotoxic. SBMP concentrations up to 0.5 mg/mL are not cytotoxic, and SBMP concentrations up to 0.25 mg/mL showed no statistical difference from the control (FIG. 9A).

While the SBMP does not induce cell death, it could inhibit cell growth. To evaluate the impact of SBMP on cell growth over time, the alamarBlue™ assay was used over 4 and 8 days to measure impacts of SBMP on human cells. In this assay, viable cells produce dehydrogenase enzymes which reduce resazurin (blue) to resorufin (pink), with higher reduction rates correlating with greater cellular activity and higher cellular viability. Resazurin was measured at days 4 and 8 of culture. After 4 days, samples containing 0.063, 0.1, 0.25, and 0.5 mg/mL SBMP statistically had the same percent of alamarBlue™ reduction as the control with no SBMP (FIG. 9B). After 8 days, there was only a significant difference between the percent reduction of alamarBlue™ of the positive control (82.7% reduction of alamarBlue™) and the 0.5 mg/mL SBMP samples (70.1% reduction of alamarBlue™) (FIG. 9B). These results show that SBMP concentrations of 0.25 mg/mL or lower had no effect on cell growth and proliferation of ADSC, while 0.5 mg/mL only affected cell growth and proliferation after 4 days of culture. The results for both cytotoxicity assays show that SBMP concentrations below 0.5 mg/mL are not cytotoxic and will not hinder cellular growth, thus use in biomedical coatings is advantageous.

Utilizing agriculture byproducts, such as SBM, for medical applications provides a sustainable way to reduce agricultural waste while harnessing the polymer's bioactive properties. To characterize SBMP, the SBMP was first produced from SBM through a process including a seven-day dialysis process. Then, the SBMP was chemically characterized using MALDI-TOF, XPS, 1H-NMR, 13C-NMR, and FTIR. These results identified a variety of compounds within the SBMP akin to lignins, tannins, and polysaccharides. These detected functional groups show that the SBMP possesses biological activity, and this was seen with the antioxidant assay. The phenolic and hydroxide groups confirmed by XPS, 1H-NMR, 13C-NMR, and FTIR correlate to the relatively high RSA exhibited by the SBMP. The presence of these compounds may also enhance the SBMP's antimicrobial activity. The SBMP exemplified high antimicrobial activity against both gram-positive and gram-negative bacteria, while displaying a more subtle fungal inhibition. The antioxidant and antimicrobial activities make the SBMP useful in a wide array of biomaterial applications. This is further supported by the cytotoxicity data. Testing up to a concentration of 0.5 mg/mL, the SBMP was not cytotoxic to the ADSC, and it supported healthy cellular growth. The SBMP can retain similar biological activities in different biomaterial compositions.

### SBMP Preparation

A feedstock is provided. The feedstock includes one or more materials derived from a beet plant (Beta sp.), such as from a beetroot or sugar beet plant (Beta vulgaris). The feedstock can comprise sugar beet molasses (SBM), and the SBM may include molasses desugarized solubles (MDS). The feedstock may be provided as a solid, liquid, paste, or syrup. The feedstock can be produced by processing beet plant biomass. Additional processing steps can remove additional sugar from the molasses to produce MDS.

The feedstock is diluted to a dilute solution and then dialyzed to produce a dialyzed solution. The dialysis step can be repeated multiple times. Water is then removed from the dialyzed solution, such as by lyophilization, to separate the produced SBMP. Alternate methods may also be used to separate the SBMP, such as chromatography or precipitation.

One limitation of current techniques for producing high molecular weight biopolymer products using complex compounds from botanical sources is that constituent compounds can include a broad range of molecules of varying composition with individual polymer chains having a widely differing degree of polymerization and molar mass. Constituent compounds can have a distribution of polymer sizes around an average value. In some cases, this heterogeneity is undesirable. Example methods can produce biopolymers, from natural botanic sources, having high molecular weights and low polydispersities. As used herein, “polydispersity index” refers to a measure of the distribution of molecular mass in a given polymer sample. The polydispersity index (PDI) is calculated by dividing the weight average molecular weight (Mw) by the number average molecular weight (Mn). Polymers can be described by molecular mass distribution; a population of particles can be described by size, surface area, and/or mass distribution;

The SBMP can comprise a composition having a molecular weight in a range from 25,000 Daltons (Da) to 125,000 Da, with a PDI in a range of 1.0 to 5.0. In some examples, the SBMP comprises a composition having a molecular weight in a range from 10,000 Da to 125,000 Da, with a PDI in a range of 1.0 to 5.0. In some examples, at least 70% of the composition by weight has a molecular weight in a range from 10,000 Daltons (Da) to 150,000 Da. In some examples, at least 80% of the composition has a molecular weight in a range from 10,000 Daltons (Da) to 150,000 Da. In some examples, 75% to 100% of the composition by weight has a molecular weight in a range from 10,000 Daltons (Da) to 150,000 Da. In some examples, at least 80% of the composition has a molecular weight in a range from 30,000 Da to 130,000 Da. In some examples, the PDI is less than 1.5, or in a range from 1.0 to 1.2. In some examples, the SBMP comprises a composition having a size peak corresponding to about 30,600 Da, with a PDI of less than 1.2. In some examples, the SBMP comprises a composition having a molecular weight size peak corresponding to about 103,500 Da, with a PDI of less than 1.2. In examples, the SBMP composition contains less than 0.5% wt./wt. sucrose.

In some examples, processing steps for producing or treating the SBMP may include one or more of: diluting, filtering, heating, cooling, emulsifying, soaking, mixing, blending, combining, reacting, oxidizing, reducing, curing, cross-linking, catalyst-mediated reaction, photoinitiated reaction, atomizing, freezing, drying, vacuum treatment, lyophilizing, isolating, purifying, chromatography, resin chromatography, size fractionation; and/or separation by charge or binding affinity.

Methods for preparing the polymer can include steps of size exclusion separation and lyophilization. In an example, a method for preparing a SBMP composition can include using dialysis. An SBM solution is prepared. A quantity of sugar beet molasses (SBM) is provided. The SBM is combined with water and mixed, for example by vortex, to produce a homogenous aqueous solution. In some examples, a ratio of the volume of the water to the volume of the SBM in the solution is greater than 1:1 and less than 10:1. A dialysis process can be used for processing. In an example, a dialysis device is prepared. A first lower end of dialysis tubing is closed and the dialysis tubing placed into a container containing deionized (DI) water. For example, the tubing can be closed by placing a dialysis clip near the bottom end of the dialysis tube, for example ½ an inch from the bottom of the dialysis tube. The tubing is positioned substantially vertically with the closed end toward the bottom of the container and upper end of the tubing slightly below the height of the container with the lower portion of the tubing submerged in the DI water. In an example, a plurality of tubing pieces may be used in the container, for example, 7 kDa snakeskin dialysis tubing (Thermo Scientific™ part #PI68700). The homogenous aqueous SBM solution is poured into the dialysis tube, with the liquid level below the top, and the second end of tubes closed. The dialysis tubes are submerged in the DI water for a separation period of time to produce a separated fraction. The water is replaced with fresh DI water periodically during the separation period. At the end of the separation period, the separated fraction is transferred to a lyophilization vessel. The separated fraction is chilled and exposed to vacuum, in some examples below 20 Pa, to lyophilize the contents of the lyophilization vessel, whereby water is removed by sublimation.

In another example method, a two-dimensional, fully aqueous fractionation can be implemented in which strong anion-exchange (AEX; Q Sepharose) provides the primary charge-based separation, followed by hydrophobic interaction chromatography (HIC; Macro-Prep HIC) as the second, orthogonal dimension applied to active AEX pools to resolve species by hydrophobicity under controlled ammonium-sulfate conditions. Fractions/sub-fractions can be rapidly desalted (ultrafiltration) before bioassays to preserve activity. Size-exclusion chromatography (SEC) can be used analytically-such as to assess dispersity/aggregation, and/or as a primary fractionation step. Removal of impurities can be performed by dialysis and/or ultrafiltration (UF), and effective impurity removal and retained bioactivity has been demonstrated. This method can be used to isolate the major classes present (polar and non-polar), remove small-molecule impurities, and can provide efficient fractionation over SEC alone.

In an example method for forming a coating or film comprising SBMP, each active pool is assayed pre- and post-film casting; and antimicrobial activity in films is assessed with an example acceptability criterion requiring ≥70% retention of solution-phase antimicrobial activity in films. In an example method, the film is formed using aqueous conditions (AEX→HIC; gentle drying; ambient temperatures; rapid desalting) to preserve labile phenolics and minimize oxidation. In some methods, complementary pools are blended and film parameters can be tuned, for example, by adjusting plasticizer level, solids content, drying rate, and/or drying temperature.

The SBMP composition can be hydrophilic, antimicrobial, antibacterial, antifungal, biodegradable, bioresorbable, and/or cytocompatible.

### Coating Examples

Medical devices can include a coating comprising at least one layer of a SBMP composition. A method for coating a medical device can include contacting SBMP to a surface of the device.

In an example method, a quantity of sugar beet molasses (SBM), optionally including molasses desugarized solubles (MDS), is processed to produce a composition comprising a sugar beet molasses polymer (SBMP). A surface of a product is prepared for coating. The composition comprising the SBMP is applied on the surface to form a substantially contiguous film comprising the SBMP. In an example the method includes one or more steps of: purification, dehydration, condensation reaction, oxidation, or curing. In some examples, one or more catalysts are used.

The coating can be applied to a medical device. In some examples, the medical device includes a material including one or more of: titanium alloy, stainless steel, cobalt chromium alloys, zirconia, carbon, ceramics, synthetic polymer, or composite materials.

The method for coating a medical device can include a pre-treatment step. Portions of a surface of the medical device can be pre-treated to customize particular coating adherence characteristics to the device. The pre-treatment of the medical device can include methods that modify the surface of the device to alter its roughness, hydrophilicity, hydrophobicity, surface charge, surface energy, biocompatibility, reactivity, and/or surface functionalization to introduce chemical functional groups to the surface of the medical device. In some examples, pre-treatment may include one or more of: mechanical or chemical abrasion, polishing, etching, chemical oxidation, pre-coating, heating, annealing, or passivation. In some examples, the step of preparing a surface includes one or more of: polishing, etching, abrading, texturing, cleaning, wetting, masking, or precoating with precoating layer.

In some examples, method includes a step of formulating an application mixture. The application mixture can comprise: the composition comprising the SBMP and a solvent. The step of formulating the application mixture can include dissolving, suspending, reconstituting, or diluting, the composition comprising the SBMP with the solvent. In some examples, the step of formulating the application mixture can include one or more of: mixing, shaking, stirring, or heating.

In some examples, the step of formulating the application mixture can include adjusting a pH. In some examples, adjusting the pH comprises adding one or more of: acetic acid, citric acid, lactic acid, hydrochloric acid, nitric acid, or other acid, or ammonia, sodium hydroxide, calcium hydroxide, calcium carbonate, potassium hydroxide, potassium bicarbonate, sodium bicarbonate, or other base.

In some examples, the step of formulating the application mixture can include adding one or more salts, including, for example, sodium chloride, potassium chloride, calcium chloride, magnesium chloride, or sodium lactate.

In some examples, the solvent comprises water and an alcohol. In some examples, the solvent comprises one or more of: ethanol, methanol, ethanol, isopropanol, butanol, ethylene glycol, polyethylene glycol, or glycerol.

The step of formulating the application mixture can include modifying a viscosity of the application mixture. In an example, the composition comprising the SBMP is provided as a powder, paste, gel, syrup, or slurry, and mixed with a solvent to produce the application mixture formulated as a liquid, gel, syrup, or slurry. In some examples, the step of adjusting the viscosity includes reducing the room-temperature viscosity to less than 100,000 milliPascal seconds (mPa s), less than 50,000 mPa s, less than 10,000 mPa s, less than 1,000 mPa s, less than 500 mPa s, less than 250 mPas, less than 100 mPa s, or less than 50 mPa s. In some examples, the step of adjusting the viscosity includes adjusting the viscosity to a viscosity in a range of 1 mPa s to 50,000 mPa s, a range of 1 mPa s to 10,000 mPa s, a range of 5 mPa s to 250 mPa s, a range of 10 mPa s to 500 mPa s, a range of 20 mPa s to 750 mPa s, or in a range of 1 mPa s to 1,000 mPa s.

The application mixture can additionally include one or more additives. In an example, the one or more additives can comprise: dye, buffer, mineral, salt, resin, surfactant, spreader, sticker, metal particle, inorganic particle, or organic compound. In an example, the additive may include inorganic particles comprising a carbonate, an oxide, a hydroxide, or a nitrate. In some examples, the step of formulating the application mixture can include combining at least one additive, whereby the application mixture comprises the additive.

In some examples, the composition is applied to a surface to form a coating. In some examples the method of applying the coating includes providing the SBMP compound in an application mixture, and the application mixture comprises a liquid, an aqueous solution, an emulsion, a foam, a gel, a syrup, a slurry, a suspension, a powder, a film, aerosolized droplets, or a spray. The application mixture may be applied to the surface of an object by an application method, including, for example, brushing, spraying, spray drying, rolling, dropping, injecting, transferring, submersion, immersion, mixing, blading, padding, and spreading.

In some examples, the coating is formed by sequentially depositing a plurality of films or layers over the surface and over earlier-deposited layers. In an example, the coating method includes depositing at least two layers, and no more than ten layers. In an example, the coating method includes depositing between one to five layers.

In some examples, the SBMP composition is combined with a second polymer. In some examples, the method includes reacting the SBMP with a polymerizing crosslinker, wherein the SBMP includes a cyclic group, and wherein the coating comprises a reaction product of the SBMP with the polymerizing crosslinker.

In an example coating method, a layer-by-layer (LbL) deposition technique can be used to form polyelectrolyte multilayers (PEM) coatings. In the process, alternating thin deposits of polycation and polyanion layers are added onto a charged surface. The electrostatic interactions of the layers help form a solid coating. Both polycations and polyanions involved in the LbL process influence the final coating properties. SBMP can be used as a polyanion layer for the PEM coating. In some examples, at least two layers of the coating comprise SBMP. In some examples, at least three layers of the coating comprise SBMP.

In some examples, PEM coatings can further control cell adhesion and inhibit bacterial growth by adding a bioactive compound at a terminal layer, wherein the terminal layer is disposed as an outermost or top layer of the coating. In some examples, the terminal layer is a polyanionic layer. In some examples, the terminal layer of the coating comprises SBMP.

An LbL method can include: pre-treating a surface to form a charged substrate; alternately contacting the surface, and any layers thereon, to the polycation coating composition, and to the polyanion coating composition, to promote layer-by-layer electrostatic self-assembly, and, thereby forming a plurality of stacked films comprising a polycationic layer, alternating with a polyanionic layer. SBMP can be used as one or more of the compositions forming the polyanion layer for the PEM coating. In some examples, the polycation layer can include one or more materials such as: chitosan, tanfloc (a condensed tannin polymer), polyethyleneimine, or collagen, including derivatives of those materials.

In some examples, the method produces a coating having a thickness equal to or greater than 5.0 nanometers (nm). In some examples, a single layer of the SBMP coating has a thickness in a range of 1.0 nm to 200 nm. In some examples, the coating comprises a plurality of layers of SBMP. In some examples, a thickness of the coating is greater than 1.0 nm, 5.0 nm, 10.0 nm, 20.0 nm, 50.0 nm, 75.0 nm, 100 nm, 200 nm, 500 nm, 600 nm, or greater than 1000 nm. In some examples, the coating has a thickness less than or equal to about 20 microns (μm) thick. In some examples, the thickness of the coating is less than 15 μm, 10 μm, 5.0 μm, 2000 nm, 1500 nm, 1000 nm, 700 nm, 400 nm, 250 nm, 150 nm, 100 nm, or less than 80 nm. The coating thickness can be substantially uniform, with an average deviation of less than 25%. In some embodiments, the coating comprises multiple layers.

In some examples, the method includes curing the as-deposited film, or plurality of films, to form the coating. The curing step can include cross-linking reactions. The curing step can include removing solvent from the film. The curing step can include one or more of: evaporation, drying, heating, baking, vacuum processing, low-pressure processing, chemical condensation, or combinations thereof. The cured film can form a substantially contiguous coating layer comprising the SBMP.

In some examples, the coating is enhanced by incorporating an additive comprising: one or more nutrients, minerals, active factors, cell adhesion peptides, growth regulators, hormones, drugs, or other chemicals. In an example, the enhancement includes one or more of: hyaluronic acid, lactic acid, alginate, chitosan, fibrin, dextran, gelatin, collagen, hydroxyapatite, calcium phosphate, calcium silicate, titanium dioxide, bone morphogenetic proteins (BMP), transforming growth factor (TGF), insulin-like growth factor (IGF), fibroblast growth factor (FGF), or vascular growth factor (VEGF). In an example, the enhancement includes at least one proteoglycan. In some examples, the coating can further include a therapeutic agent.

In some examples, the additive is incorporated into one or more layers during the coating process.

In some examples, the additive is added in a functionalizing step after formation of the coated layer.

In an example, the composition further comprises a coating enhancer to modify the rheometric properties and viscosity of the composition. The coating enhancer may include starch, amylose, amylopectin, dextrin, maltodextrin, polydextrose, syrup, cellulose, gum Arabic, gum tragacanth, gum karaya, mesquite gum, galactomannan, pectin, carrageenan, alginate, dextran, xanthan, gellan, silk protein, casein, gelatin, gluten, fatty acids, fatty alcohols, wax, glycerol, glyceride, phospholipid, PVP, paraffin, or a solgel. In an example method, the composition substantially coats a surface and forms a dried residue on the surface. In an example method, the composition may be applied to a surface with a coating enhancer to form a coating, film, or residue on the surface.

In some examples, the coating can be applied to a device or product. In some examples, the coating is applied to a surface of a medical device. The surface can include a portion of an exterior of the device.

A medical device provided for use in a method may include one or more of: an orthopedic device, a cardiovascular device, a tissue scaffold, or an endoprosthetic. Catheters, stents, shunts, grafts, pacemakers, defibrillators, wound dressings, bandages, insulin pumps, and implants are contemplated as medical devices employed in a method of the invention. Synthetic implants, such as breast, penile, dental, cochlear implants, and including stabilizing structures, such as pins, screws, plates, and mesh, and synthetic devices, such as knee, hip, and shoulder prostheses, are contemplated as medical devices for use with described methods.

A medical device may have a structure made of metal, ceramic, and/or polymer plastics. In some examples, the structure can include one or more of: titanium, stainless steel, cobalt chrome, bioglass, apatite, calcium phosphate, calcium sulfate, aluminum, zirconia, polyurethane, polyacrylate, polyester, and/or silicone.

In one non-limiting example, the coating is applied to a surgical steel hip replacement device, and the coating is applied to a femoral stem portion of the device, configured to be positioned adjacent to the femur, thereby providing a SBMP coating between the metal of the implant and the segment of bone where it is implanted. The SBMP coating can provide a biodegradable surface, promoting bone growth and cell adhesion, while also reducing infection risk. In an example, the medical device is treated prior to coating with an agent that enhances the adherence of the coating to the device. Such an agent can include, for example, calcium chloride or calcium phosphate.

In an example, metal-based bone and joint implants can benefit from a biopolymer coating comprising SBMP. Some metals, such as titanium, are biocompatible and used in osteopathic medicine in the manufacturing of artificial joints. Metals are susceptible to erosion and infection and lack biological activity. When metals are used as an implant, problems can arise when contacting associated bone and tissues, such as static stresses that can lead to bone loss at the site of implantation. These metal joints and bone implants can be coated in polymeric material to reduce infection risk and reduce inflammatory responses that can be triggered by joint implants. The coating can optionally include therapeutic agents, such as growth factors or drugs to mitigate risk of infection and rejection. Coatings and coating methods that provide adherent, mechanically stable, biocompatible, and antimicrobial properties can improve osseointegration of an implant device, leading to longer service life for implanted devices, greater long-term surgical success rates and better quality of life for patients.

In some embodiments, the coating produces a surface that is substantially hydrophilic, whereby a droplet contact angle is equal to or less than 90 degrees. In some examples, the contact angle is between 20 degrees and 80 degrees.

Cellular recognition of natural polymers derived from animal sources can initiate an immune response in sensitive patients; thus, replacing an animal-derived material, like fibrin, gelatin, or collagen, with a botanically-derived or plant-sourced polymer, such as SBMP, in medical devices and implants can reduce risks of implant rejection and immune-mediated inflammation. In some examples, the coating has no animal-derived material. In some examples, the coating has less than 1% animal-derived material by dry weight.

### Hydrogels, Fillers, Growth Media, Cements, Scaffolds, and Nanoclays

In tissue engineering, a highly porous artificial extracellular matrix supports and guides cell growth and tissue regeneration. Natural and synthetic biodegradable polymers have been used to create such scaffolds. Hydrogel wound dressings can be designed to facilitate oxygen diffusion to accelerate healing, prevent infection, retain moisture, and promote cellular proliferation. Injectable fillers for soft tissues can be provided as gels or suspensions, and can be used to treat tendon injuries, promote gum regeneration, and for other medical and cosmetic treatments.

Provided are methods and compositions for incorporating a SBMP composition into a biomedical product, such as a hydrogel, filler, growth media, cement, scaffold, or nanoclay. The SBMP composition can be blended with other materials to form a composite material. The SBMP composition can be incorporated into an amorphous product, like bone cement or endodontic dental filling. In some examples, the SBMP composition is bioresorbable. In some examples, the SBMP composition is dissolved into an aqueous liquid. In some examples, the SBMP composition is powdered, such as by lyophilization and grinding, and mixed with additional materials to form the biomedical product.

In an example, the SBMP composition is combined with an initiator, an accelerator, and one or more additional polymers, such as resin or acrylate-based synthetic polymers or polymer precursors, to form an amorphous liquid or paste that cures or hardens into a solid or a colloid. In some examples, the biomedical product comprises between 0.10% to 10.0% dry weight SBMP.

In an example, the SBMP composition is combined with a second polymer to form a hydrogel. Natural polymers for hydrogel preparation can include hyaluronic acid, chitosan, heparin, alginate, gelatin, or fibrin. In some examples, the hydrogel has less than 1% animal-derived material by dry weight.

In an example, the SBMP composition formed into a three-dimensional porous structure to form a tissue scaffold. In an example, living animal cells are adhered to the SBMP of the tissue scaffold.

In an example, the SBMP composition is combined with a second material, optionally a second polymer, to form a tissue scaffold. In some examples, a second material can include one or more of: collagen, cellulose, silk fibroin, keratin, gelatin, chitosan, agar, agarose, agaropectin, alginate, or another natural or synthetic polymer. A synthetic polymer can include, for example, poly(lactic acid) (PLA), polycaprolactone (PCL), polyurethane (PU), poly(lactic-co-glycolic acid) (PLGA), poly(L-lactide) (PLLA), or poly(ethylene-co-vinylacetate) (PEVA). A blend of SBMP with another polymer, for a scaffold matrix can provide biomimetic structure, good mechanical strength, high spatial interconnectivity, high porosity, controlled alignment, and favorable bioactivity.

In some examples, a ratio of SBMP to the second material is equal to or greater than: 1:100, 1:50, 1:10, or 1:1. In some examples, a ratio of SBMP to the second material is less than 100:1, 50:1, 10:1, or 1:1. In some examples, a ratio of SBMP to the second material is in a range of 1:10 to 10:1.

In some examples, the SBMP composition or coating is used as a substrate, scaffold, or growth media for in vitro tissue culture intended for tissue transplant. Due to its botanical source, use of the SBMP can reduce risk of immune response to the tissue.

In some examples, the SBMP composition or coating is used on a medical implant in vivo to promote tissue repair.

In some examples, the SBMP composition or coating is provided in a wound dressing or bandage. In some examples, a composite biomaterial comprises: SBMP and at least one second material, wherein the second material comprises at least one of: collagen, cellulose, silk fibroin, keratin, gelatin, chitosan, alginate, or another natural or synthetic polymer.

In some examples, a hydrogel comprises: SBMP and at least one proteoglycan. In some examples, the coating or hydrogel has less than 1% animal-derived material wt/wt.

Materials and methods using SBMP are described. A method of coating a surface of an object is provided. Antimicrobial coatings are provided. Methods of using and producing antimicrobial compositions are provided. Formulations and methods are provided for inhibiting and controlling infection. Compositions and methods are provided to promote wound healing and facilitate recovery from procedures for placing medical implants. Described compositions can include SBMP. Methods for producing coatings comprising antimicrobial compositions are provided.

Methods and compositions are described for SBMP produced from molasses. Polymer compounds are provided. The polymers can be formulated as hydrogels, fillers, cements, scaffolds, nanoclays, and/or coating compositions for use on medical devices. The polymer compositions can be antimicrobial. In some examples, the biopolymer compositions are bioresorbable. The polymer compositions can promote cell adhesion.

### Coatings and Compositions for Systems, Horticulture, Animal Care, Food, and Cosmetics

Materials and methods using SBMP are described for use in systems and products. In some examples, the SBMP composition or coating is provided on a surface of, or incorporated into a material for: a medical device, a veterinary product, an air filter, an article of clothing, an article of hygiene, a building material, a face mask, a drink container, or a food package.

In some examples, the SBMP composition or coating is used in a component of an HVAC system, such as an air filter or humidifier.

In some examples, the SBMP composition or coating is used in a component of a liquid management system, such as a coating on an interior surface of a water pipe, fluid pump, filter, or reservoir.

In some examples, the SBMP composition or coating is used in a substrate media for agriculture or horticulture, such as with soil or soil substitute, hydroponic substrate, growth container, or petri dish. In some examples, the SBMP composition or coating is used in a horticultural composition, such as a pruning dressing, plant or tree wound treatment, grafting sealant, a mushroom substrate, a seed coating, or bark protectant.

In some examples, the SBMP composition or coating is incorporated into articles for use in the care of animals, including pets, livestock, aquaculture, pisciculture, and insect farming. In some examples the SBMP composition or coating is provided on a surface of, or incorporated into a material for: a water trough, a water jar, a feeding trough, a food dish, a feeding container, a tray, a hoof protectant, a beehive, a bait jar, an aquarium, aquarium substrate, a net, an enclosure material, pond liner, floor mat, bedding, or litter.

In some examples, the SBMP composition or coating is incorporated into articles for use in food processing and delivery. In an example, the SBMP composition or coating is generally recognized as safe (GRAS) for uses in the food and cosmetics industry. In an example, it is used in food packaging. In an example, a SBMP is provided as a coating on a fiber-based food tray, whereby the coated tray is biodegradable. In an example, it is used in beverage fermentation or culturing, such as in a container for making or processing beer, wine, vinegar, or kombucha.

In some examples, the SBMP composition or coating is used as a substrate, scaffold, or growth media for in vitro cell or tissue culture for artificial or synthetic meat products.

In some examples, the SBMP composition or coating is incorporated into articles for use in human cosmetics or hygiene. In an example, it is used in packaging. In another example, it is used in a cleaning wipe. In some examples, a method of inhibiting microbial growth comprises: contacting a surface with an effective amount of an SBMP composition. In some examples, the contacting comprises a rinse, soak, or spray. In some examples, the contacting comprises forming a film on the surface. In some examples, a method of treating comprises contacting a product with an effective amount of an SBMP composition. In an example, the SBMP composition is incorporated into an emulsion, a suspension, a foam, or a cream. In some examples, a method of inhibiting microbial growth comprises formulating the product with an effective amount of an SBMP composition.

The compounds can be incorporated as an additive to cosmetic and personal care products, for example, in a solution or emulsion as an antioxidant or antimicrobial. In some examples, the SBMP composition is used as an antioxidant additive. In some examples, the SBMP composition is used as an antimicrobial additive. In some examples, the additive is provided in an aqueous solution, an emulsion, or a gel. In some examples, a product comprising the SBMP composition includes: a healthcare product, hygiene product, cosmetics product, or animal care product.

A method of controlling microbial growth can include formulating a product to include the SBMP composition. A method of providing an antioxidant can include formulating a product to include the SBMP composition. In some examples, a product is provided comprising the SBMP composition. In some examples, the product includes one or more of: a healthcare product, a hygiene product, a cosmetics product, or an animal care product. In a few specific examples, a product comprising SBMP can include one or more of: mascara, eyeliner, sunscreen, or lotion. In an example, a product can include the SBMP composition at a concentration in a range of 1.0% to 50% dry weight. In some examples, a product can include the SBMP composition at a dry weight (wt/wt) concentration greater than 1.0%, 2.0%, 3.0% 5.0%, 8.0%, 10%, 15%, 20%, 25%, 30%, 40%, or 45%. In some examples, a product can include the SBMP composition at a dry weight (wt/wt) concentration in a range of 5% to 30%. In some examples, the product contains no animal byproducts. In some examples, the product contains no artificial preservatives. In some examples, the product is shelf-stable at room temperature for 3-12 months. In some examples, the product does not contain any of one or more of: parabens, phenoxyethanol, propionate, pyridinethione, formaldehyde, urea, benzyl alcohol, benzalkonium chloride, potassium sorbate, sodium metabisulfite, or sodium benzoate.

In some examples, a method for formulating a cosmetic product is provided wherein the SBMP composition is formulated into cosmetic product and the cosmetic product includes at least one of: colorant, wax, resin, emulsifier, essential oil, mineral powder, fragrance. In some examples the SBMP composition is present in the product at a level in a range of 1.0% to 30% dry weight percent. In an example, the cosmetic product wax comprises one or more of: hydrogenated olive esters, beeswax, carnauba wax, jojoba wax, rice wax, candelilla wax, lanolin wax, orange peel wax, bayberry wax, soy wax, and sunflower wax.

In some examples, the SBMP composition or coating is combined with one or more appearance modifiers, such as: dye, pigment, resin, nanoparticles, light reflective material, light absorptive material, photochromatic material, opacity modifier, flattener, or de-glossing agent. In an example, a paint product can include the SBMP composition, at least one appearance modifier, and one or more: catalysts, thickeners, stabilizers, emulsifiers, texturizers, adhesion promoters, UV stabilizers, biocides, diluent, solvent, or binder. In an example, a painted coating can include the SBMP composition at a concentration in a range of 1.0% to 50% dry weight. In some examples, a coating or film can include the SBMP composition at a dry weight (wt/wt) concentration greater than 1.0%, 2.0%, 3.0% 5.0%, 8.0%, 10%, 15%, 20%, 25%, 30%, 40%, or 45%. In some examples, a product can include the SBMP composition at a dry weight (wt/wt) concentration in a range of 5% to 30%.

Certain embodiments of the devices, apparatuses, and methods disclosed herein are described in the above examples. It should be understood that these examples, while indicating particular embodiments, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure, and to adapt a particular situation or material to the teachings of the disclosure without departing from the essential scope thereof.

What is claimed is:

1. A biopolymer composition comprising SBMP, wherein the SBMP is produced from sugar beets.

2. The biopolymer composition of claim 1, wherein at least 80% of the composition has a molecular weight in a range from 10,000 Daltons (Da) to 150,000 Da.

3. The biopolymer composition of claim 1, wherein the SBMP has an average molecular weight in a range from 25,000 Da to 125,000 Da, with a polydispersity index in a range from 1.0 to 1.5.

4. The biopolymer composition of claim 1, wherein the biopolymer, at 1 mg/mL, has a radical scavenging activity (RSA) equivalent equal to or greater than 0.15 mg/mL ascorbic acid, using an assay of 2,2-diphenyl-1-(2,4,6-trinitrophenyl) hydrazyl (DPPH) with ascorbic acid as the reference standard.

5. A cosmetic product comprising:
the SBMP composition of claim 1; and
at least one of: colorant, wax, resin, emulsifier, essential oil, mineral powder, or fragrance; and
wherein the SBMP composition is present in the product at a level in a range of 1.0% to 30% dry weight.

6. A coating for a product comprising the SBMP composition of claim 1; wherein the coating has a coating surface that is substantially hydrophilic, whereby a droplet contact angle is less than or equal to 90 degrees.

7. The coating of claim 6, wherein the product includes at least one of: an orthopedic device, a cardiovascular device, a tissue scaffold, an endoprosthetic, a catheter, a stent, a shunt, a graft, a pacemaker, a defibrillator, a wound dressing, a bandage, an insulin pump, or an implant.

8. A method of coating a surface of an object, the method comprising:
providing a coating composition that includes a polymer comprising SBMP; and
contacting the coating composition to a surface of the object.

9. The method of claim 8, wherein the method comprises a layer-by-layer (LbL) coating method, comprising:
providing a polycation coating composition;
providing a polyanion coating composition comprising the SBMP;
pre-treating the surface to form a charged substrate;
alternately contacting the surface, and any layers thereon, to the polycation coating composition, and to the polyanion coating composition, to promote layer-by-layer electrostatic self-assembly, and, thereby forming a plurality of stacked films comprising a polycationic layer, alternating with a polyanionic layer; wherein:
the coating comprises the plurality of stacked films;
the plurality of stacked films comprises at least three layers; and
each layer has a thickness in a range of 1.0 nm to 100 nm.

10. The method of claim 8, wherein the method further comprises a functionalizing step, wherein the functionalizing step incorporates at least one additive to the coating; and wherein the at least one additive comprises one or more of: hyaluronic acid, lactic acid, alginate, chitosan, fibrin, dextran, gelatin, collagen, hydroxyapatite, calcium phosphate, calcium silicate, titanium dioxide, bone morphogenetic proteins (BMP), transforming growth factor (TGF), insulin-like growth factor (IGF), fibroblast growth factor (FGF), or vascular growth factor (VEGF).

11. A method of inhibiting microbial growth comprising:
providing an effective amount of an SBMP composition.

12. The method of claim 11, wherein:
the SBMP composition SBMP is derived from sugar beets;
70% to 100% of the composition has a molecular weight in a range from 10,000 Daltons (Da) to 150,000 Da.

13. The method of claim 11, wherein the SBMP composition is formulated in a cosmetic product and present at a level in a range of 1.0% to 30% dry weight percent.
